(19) United States
(12) Patent Application Publication
(10) Pub. No.: US 2025/0019640 A1
(43) Pub. Date: Jan. 16, 2025

(54) CULTURE METHOD FOR HIGH-CONCENTRATION HAEMATOCOCCUS PLUVIALIS

(71) Applicant: Algae Origin Inc., Newton, MA (US)

(72) Inventor: Bingchun CHANG, Newton, MA (US)

(73) Assignee: Algae Origin Inc., Newton, MA (US)

(21) Appl. No.: 18/219,731

(22) Filed: Jul. 10, 2023

(51) Int. Cl. C12N 1/12

(52) U.S. Cl. CPC C12N 1/12

(57) ABSTRACT

A culture method for high-concentration Haematococcus pluvialis includes the following steps: 1) inoculating a Haematococcus pluvialis algae into a first culture medium for culturing for 7-10 days, collecting algal cells, inoculating the algal cells into a new first culture medium for culturing until a dry weight concentration of the algal cells in a Haematococcus pluvialis liquid obtained by culturing is 2.0 g/L or more, and collecting the algal cells, namely motile vegetative cells; 2) inoculating the motile vegetative cells into a second culture medium for culturing for 13-15 days to obtain nonmotile vegetative cells with aged cell walls; and 3) inoculating the nonmotile vegetative cells into a third culture medium for culturing for 10-14 days, inducing the generation of astaxanthin until a content of the astaxanthin is 6% or more, and finishing the culture.

### TECHNICAL FIELD

The present invention belongs to the technical field of algae culture, and particularly relates to a culture method for high-concentration Haematococcus pluvialis.

### BACKGROUND

Haematococcus pluvialis is a unicellular green alga, which can accumulate a large amount of astaxanthin to be red, and the content of astaxanthin can reach 3-5% of the dry weight of cells. Haematococcus pluvialis is a main source of natural astaxanthin at present. The life history of Haematococcus pluvialis is divided into two stages of vegetative cells and akinetes: under the conditions of proper environment and rich nutrition, Haematococcus pluvialis rapidly grows, divides and propagates to generate a large number of motile vegetative cells with flagella; when environmental conditions become unfavorable, the motile cells lose flagella and become nonmotile vegetative cells; under the stimulation of continuous adverse environmental conditions, such as high light, high salt, nutritional starvation, etc., the vegetative cells will not continue to divide and propagate, and form red akinetes by accumulating a large amount of astaxanthin within the cells to combat the adverse environmental conditions.

According to the physiological characteristics of Haematococcus pluvialis, a three-step method is usually adopted for large-scale culture, namely motile vegetative cell culture, nonmotile vegetative cell culture, and induction culture. The culture of vegetative cells and the culture of nonmotile vegetative cells are usually carried out indoors, and continuous production can be carried out all year round due to controllable conditions such as illumination, temperature, and the like. In order to reduce the resource investment, most enterprises choose to carry out induction culture outdoors, but due to the uncontrollable nature of the external environment, the outdoor culture conditions are greatly influenced by weather, and if severe environmental conditions such as continuous rainy weather, frost, temperature reduction and the like occur, the yield of astaxanthin can be greatly reduced, and even production interruption occurs. Although the indoor vegetative cell culture is not influenced, the production has to be stopped because the outdoor induction culture cannot be carried out, so that the problems of low equipment utilization rate, idle workers and the like are caused. In addition, when the outdoor culture conditions recover, Haematococcus pluvialis is slow in growth and cannot supply sufficient vegetative cells for induction culture in time, which also results in a reduction in yield. That is to say, the traditional culture process for Haematococcus pluvialis often causes overall production stoppage when the weather conditions are not suitable for outdoor induction culture, and cannot provide sufficient vegetative cells in time to quickly recover production when the weather conditions recover to be normal, so that the yield is reduced, and the production is unstable.

### SUMMARY

In view of this, the present invention aims to provide a culture method for high-concentration Haematococcus pluvialis, and with the culture method, astaxanthin is induced outdoors after accumulation of high concentration biomass of Haematococcus pluvialis in a laboratory.

The present invention provides a culture method for high-concentration Haematococcus pluvialis, comprising the following steps:
1) inoculating a Haematococcus pluvialis algae into a first culture medium for culturing for 7-10 days, collecting algal cells, inoculating the algal cells into a new first culture medium for culturing until a dry weight concentration of the algal cells in a Haematococcus pluvialis liquid obtained by culturing is 2.0 g/L or more, and collecting the algal cells, namely motile vegetative cells,
wherein the first culture medium is an improved Bold's Basal Medium (BBM) culture medium, and a concentration of NaNO₃ in the culture medium is 1.3-1.7 g/L;
2) inoculating the motile vegetative cells obtained in step 1) into a second culture medium for culturing for 13-15 days to obtain nonmotile vegetative cells with aged cell walls, wherein the second culture medium is a simplified BBM culture medium, and the simplified BBM culture medium does not comprise NaNO₃;
3) inoculating the nonmotile vegetative cells obtained in step 2) into a third culture medium for culturing for 10-14 days, inducing the generation of astaxanthin until a content of the astaxanthin in algal powder is 6% or more, and finishing the culture,
wherein the third culture medium is an improved BBM culture medium, and a concentration of NaCl in the culture medium is 1.3-1.7 g/L.

Preferably, the first culture medium uses water as a solvent and comprises the following components in the following concentrations: 1.3-1.7 g/L NaNO₃, 0.7-0.8 g/L MgSO₄ 7H₂O, 0.2-0.3 g/L NaCl, 0.7-0.8 g/L K₂HPO₄, 1.7-1.8 g/L KH₂PO₄, 0.2-0.3 g/L CaCl₂·2H₂O, 0.11-0.12 g/L H₃BO₃, 0.8-1.2 mL/L trace element liquid, 0.8-1.2 mL/L EDTA solution, and 0.8-1.2 mL/L Fe solution.

Preferably, the second culture medium uses water as a solvent and comprises the following components in the following concentrations: 0.7-0.8 g/L MgSO₄·7H₂O, 0.2-0.3 g/L NaCl, 0.7-0.8 g/L K₂HPO₄, 1.7-1.8 g/L KH₂PO₄, 0.2-0.3 g/L CaCl₂·2H₂O, 0.11-0.12 g/L H₃BO₃, 0.8-1.2 mL/L trace element liquid, 0.8-1.2 mL/L EDTA solution, and 0.8-1.2 mL/L Fe solution.

Preferably, the third culture medium uses water as a solvent and comprises the following components in the following concentrations: 0.4-0.6 g/L NaNO₃, 0.7-0.8 g/L MgSO₄·7H₂O, 1.3-1.7 g/L NaCl, 0.7-0.8 g/L K₂HPO₄, 1.7-1.8 g/L KH₂PO₄, 0.2-0.3 g/L CaCl₂·2H₂O, 0.11-0.12 g/L H₃BO₃, 0.8-1.2 mL/L trace element liquid, 0.8-1.2 mL/L EDTA solution, and 0.8-1.2 mL/L Fe solution.

Preferably, the water has a conductivity of 10 S/m or less.

Preferably, an illumination intensity during the culture of the motile vegetative cells in step 1) is controlled at 30-100 μmol/m²; the ambient temperature is 24-26° C.; a volume ratio of CO₂ introduced into the system to air introduced into the system is 0.5-1:99.5-99.

Preferably, an illumination intensity during the culture of the nonmotile vegetative cells in step 2) is controlled at 100-125 μmol/m²; the ambient temperature is 24-26° C.; a volume ratio of CO₂ introduced into the system to air introduced into the system is 1-2:99-98.

Preferably, an illumination intensity during the induction of the generation of astaxanthin in step 3) is controlled at 125-300 μmol/m²; the ambient temperature is 24-26° C.; a volume ratio of CO₂ introduced into the system to air introduced into the system is 1-2:99-98.

Preferably, the illumination intensity is controlled by the aid of a light conversion film.

Preferably, the Haematococcus pluvialis is cultured using pipeline culture; in the process of the culture of the motile vegetative cells, a bottom of the used pipeline is conical, and a ventilation pipe is arranged in the pipeline.

Compared with the prior art, the present invention has the following beneficial effects: the culture method for high-concentration Haematococcus pluvialis provided by the present invention, by adjusting the components and the content of culture media in different culture stages, at the stage of the culture of the motile vegetative cells, increases the content of sodium nitrate to 1.3-1.7 g/L from the original 0.5 g/L, increases the cell biomass accumulation, and increases mass culture at the stage of green cells in a laboratory to the maximum extent; at the stage of the culture of the nonmotile vegetative cells, sodium nitrate is not added to the culture medium, so that the conversion of the nonmotile vegetative cells to the motile vegetative cells is promoted, the cell wall aging is promoted, the capacity of resisting the external severe environment is increased, and the assistance is provided for the survival of Haematococcus pluvialis in the natural environment; at the stage of the induction of the generation of astaxanthin, sodium chloride is added to promote the generation of astaxanthin, increase the accumulation amount of astaxanthin, and shorten the induction period.

Furthermore, the present invention controls the illumination intensity by the aid of a light conversion film, promotes the cell biomass to be accumulated to the highest limit capable of being accumulated, shortens the cell cycle, and improves the accumulation of astaxanthin.

The present invention, by changing the culture medium at different culture stages, adjusting the culture period, and controlling the illumination intensity, can realize uninterrupted production, increase mass culture at the stage of green cells in a laboratory to the maximum extent, maximize the accumulation amount of astaxanthin at the stage of astaxanthin induction to greatly shorten the whole culture period, improve the utilization rate and the productivity of equipment, increase the accumulation amount of astaxanthin, and thus improve the overall stability of the production quality.

### BRIEF DESCRIPTION OF THE DRAWINGS

FIG. 1 is a schematic view of pipeline culture of motile vegetative cells.

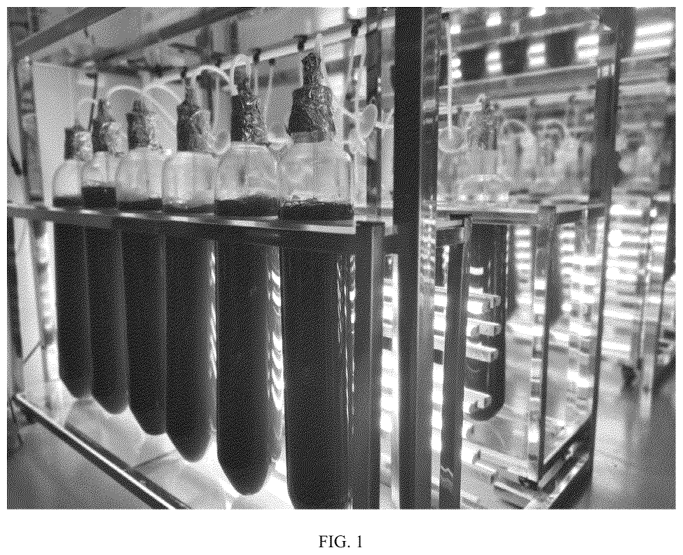

FIG. 2 is a schematic view showing the arrangement of a light conversion film at the stage of the culture of the motile vegetative cells and at the stage of the induction of the generation of astaxanthin.

FIG. 3 is a schematic view of the stage of the large-scale outdoor induction of the generation of astaxanthin.

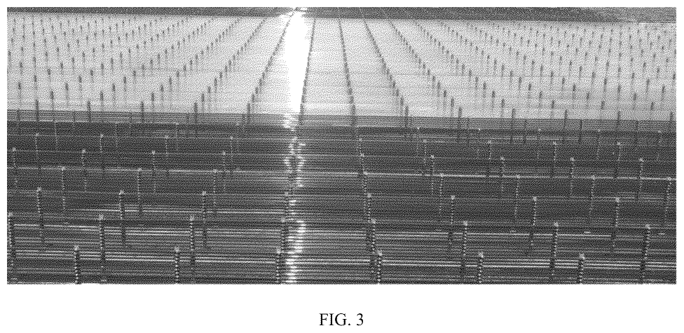

### DETAILED DESCRIPTION OF THE EMBODIMENTS

The present invention provides a culture method for high-concentration Haematococcus pluvialis, comprising the following steps: 1) inoculating a Haematococcus pluvialis algae into a first culture medium for culturing for 7-10 days, collecting algal cells, inoculating the algal cells into a new first culture medium for culturing until a dry weight concentration of the algal cells in a Haematococcus pluvialis liquid obtained by culturing is 20 g/L or more, and collecting the algal cells, namely motile vegetative cells, wherein the first culture medium is an improved BBM culture medium, and a concentration of NaNO₃ in the culture medium is 1.3-1.7 g/L; 2) inoculating the motile vegetative cells obtained in step 1) into a second culture medium for culturing for 13-15 days to obtain nonmotile vegetative cells with aged cell walls, wherein the second culture medium is a simplified BBM culture medium, and the simplified BBM culture medium does not comprise NaNO₃; and 3) inoculating the nonmotile vegetative cells obtained in step 2) into a third culture medium for culturing for 10-14 days, inducing the generation of astaxanthin until a content of the astaxanthin is 6% or more, and finishing the culture, wherein the third culture medium is an improved BBM culture medium, and a concentration of NaCl in the culture medium is 1.3-1.7 g/L.

In the present invention, the Haematococcus pluvialis algae were inoculated into a first culture medium for culturing for 7-10 days, and algal cells were collected. In the present invention, the Haematococcus pluvialis JNU35 described in Chinese patent CN110343616B is preferably used as the Haematococcus pluvialis algae. In the present invention, the first culture medium is preferably an improved BBM culture medium, and a concentration of NaNO₃ in the culture medium is 1.3-1.7 g/L, preferably 1.4-1.6 g/L, and more preferably 1.5 g/L; further preferably, the first culture medium uses water as a solvent and comprises the following components in the following concentrations: 1.3-1.7 g/L NaNO₃, 0.7-0.8 g/L MgSO₄·7H₂O, 0.2-0.3 g/L NaCl, 0.7-0.8 g/L K₂HPO₄, 1.7-1.8 g/L KH₂PO₄, 0.2-0.3 g/L CaCl₂·2H₂O, 0.11-0.12 g/L H₃BO₃, 0.8-1.2 mL/L trace element liquid, 0.8-1.2 mL/L EDTA solution, and 0.8-1.2 mL/L Fe solution; more preferably, 1.5 g/L NaNO₃, 0.75 g/L MgSO₄·7H₂O, 0.25 g/L NaCl, 0.75 g/L K₂HPO₄, 1.75 g/L KH₂PO₄, 0.25 g/L CaCl₂·2H₂O, 0.114 g/L H₃BO₃, 1.0 mL/L trace element liquid, 1.0 mL/L EDTA solution, and 1.0 mL/L Fe solution. In the present invention, a concentration of sodium nitrate in the first culture medium is limited to 1.3-1.7 g/L. The concentration range is the optimal nitrogen concentration condition for culturing Haematococcus pluvialis, and the concentration range can enable the cell density of the Haematococcus pluvialis to reach or approach the maximum after the nitrogen source is used up, and meanwhile, the Haematococcus pluvialis can directly enter the sodium nitrate nutrition adding concentration at the astaxanthin accumulation stage at the fastest speed. In the present invention, the first medium is preferably used after sterilization, and the sterilization conditions are preferably: 120-130° C., 0.1-0.2 MPa, and sterilizing for 10-20 min; preferably, after the sterilization is finished, naturally cooling is performed, and the temperature is cooled to 20-25° C. for inoculation; the inoculation amount of the Haematococcus pluvialis is preferably 0.3-0.7 g/L, more preferably 0.4-0.6 g/L, and most preferably 0.5 g/L on a dry weight basis.

According to the present invention, the algal cells are collected after 7-10 days of culture; the collection of the algal cells is preferably carried out by means of precipitation, and the precipitation time is preferably 4-6 h. According to the present invention, after the algal cells are collected and obtained, the algal cells are inoculated into a new first culture medium for culturing until a dry weight concentration of the algal cells in the Haematococcus pluvialis liquid obtained by culturing is 20 g/L or more, and the algal cells are collected, namely motile vegetative cells.

In the present invention, the Haematococcus pluvialis is cultured using pipeline culture; in the process of the culture of the motile vegetative cells, a bottom of the used pipeline is conical, and a ventilation pipe and a sampling pipe arranged in the pipeline; further preferably, a special cylindrical culture pipe is adopted, the diameter of an upper opening of the special cylindrical culture pipe is preferably 5.5-6.5 cm, and the diameter of a lower opening of the special cylindrical culture pipe is preferably 11.5-12.5 cm; the ventilation pipe is preferably 4-6 cm higher than the culture pipe; an outer diameter of the ventilation pipe is preferably 5.5-6.5 mm, and an inner diameter of the ventilation pipe is preferably 2.5-3.5 mm; the sampling pipe is set up at the upper opening of the cylindrical culture pipe, the bottom of the sampling pipe is located below half of the liquid level of the culture medium, an outer diameter of the sampling pipe is preferably 2.5-3.5 mm, and an inner diameter is preferably 0.5-1.5 mm. In the present invention, the positions of the ventilation pipe and the culture pipe are fixed; the distance between the ventilation pipe and the bottom of the special cylindrical culture pipe is 2-3 mm, so that ventilation is ensured. In the present invention, the special cylindrical culture pipe is preferably sterilized before use.

According to the present invention, an illumination intensity during the culture of the motile vegetative cells is preferably controlled at 30-100 μmol/m², and more preferably 50 μmol/m²; the ambient temperature is preferably 24-26° C., and more preferably 25° C.; a volume ratio of CO₂ introduced into the system to air introduced into the system is preferably 0.5-1:99.5-99.

According to the present invention, after the motile vegetative cells are obtained, the motile vegetative cells obtained are inoculated into a second culture medium for culturing for 13-15 days to obtain nonmotile vegetative cells with aged cell walls. In the present invention, the second culture medium does not comprise NaNO₃; the second culture medium uses water as a solvent and preferably comprises the following components in the following concentrations: 0.7-0.8 g/L MgSO₄·7H₂O, 0.2-0.3 g/L NaCl, 0.7-0.8 g/L K₂HPO₄, 1.7-1.8 g/L KH₂PO₄, 0.2-0.3 g/L CaCl₂·2H₂O, 0.11-0.12 g/L H₃BO₃, 0.8-1.2 mL/L trace element liquid, 0.8-1.2 mL/L EDTA solution, and 0.8-1.2 mL/L Fe solution; more preferably, 0.75 g/L MgSO₄·7H₂O, 0.25 g/L NaCl, 0.75 g/L K₂HPO₄, 1.75 g/L KH₂PO₄, 0.25 g/L CaCl₂·2H₂O, 0.114 g/L H₃BO₃, 1.0 mL/L trace element liquid, 1.0 mL/L EDTA solution, and 1.0 mL/L Fe solution. Sodium nitrate is not added, so that a nitrogen deficiency environment is created, and the generation of astaxanthin is induced.

In the present invention, an illumination intensity during the culture of the nonmotile vegetative cells is controlled at 100-125 μmol/m²; the ambient temperature is 24-26° C., preferably 25° C.; a volume ratio of CO₂ introduced into the system to air introduced into the system is 1-2:99-98. In the present invention, the illumination intensity is controlled by the aid of a light conversion film, which is consistent with the stage of the culture of the motile vegetative cells described above, and specific details will not be repeated here.

According to the present invention, after the nonmotile vegetative cells are obtained, the nonmotile vegetative cells obtained are inoculated into a third culture medium for culturing for 10-14 days, and the generation of astaxanthin is induced until a content of the astaxanthin is 6% or more, and the culture is finished. In the present invention, a concentration of NaCl in the third culture medium is 1.3-1.7 g/L, preferably 1.4-1.6 g/L, and more preferably 1.5 g/L. The third culture medium uses water as a solvent and preferably comprises the following components in the following concentrations: 0.4-0.6 g/L NaNO₃, 0.7-0.8 g/L MgSO₄·7H₂O, 1.3-1.7 g/L NaCl, 0.7-0.8 g/L K₂HPO₄, 1.7-1.8 g/L KH₂PO₄, 0.2-0.3 g/L CaCl₂·2H₂O, 0.11-0.12 g/L H₃BO₃, 0.8-1.2 mL/L trace element liquid, 0.8-1.2 mL/L EDTA solution, and 0.8-1.2 mL/L Fe solution; more preferably, 0.5 g/L NaNO₃, 0.75 g/L MgSO₄·7H₂O, 1.5 g/L NaCl, 0.75 g/L K₂HPO₄, 1.75 g/L KH₂PO₄, 0.25 g/L CaCl₂·2H₂O, 0.114 g/L H₃BO₃, 1.0 mL/L trace element liquid, 1.0 mL/L EDTA solution, and 1.0 mL/L Fe solution.

According to the present invention, an illumination intensity during the induction of the generation of astaxanthin is preferably controlled at 125-300 μmol/m²; the ambient temperature is preferably 24-26° C., and more preferably 25° C.; a volume ratio of CO₂ introduced into the system to air introduced into the system is preferably 1-2:99-98.

In the present invention, the illumination intensity is controlled by the aid of a light conversion film; the light conversion film comprises a combined film consisting of an opaque film in the bottom layer, a light-reflecting film in the middle layer, and a light conversion film in the upper layer; the light conversion film in the upper layer is preferably the light conversion film described in Chinese Patent CN115261018A.

In the present invention, the trace element liquid in the first culture medium, the second culture medium, and the third culture medium described above preferably use water as a solvent, and comprise the following components on a basis of 1 L: 8.82 g ZnSO₄·7H₂O, 1.44 g MnCl·4H₂O, 0.71 g MoO₃, 1.57 g CuSO₄·5H₂O, 0.49 g Co(NO₃)₂ 6H₂O, and 1.0 mL H₂SO₄; the EDTA solution uses water as a solvent, and comprises the following components on a basis of 1 L: 50 g EDTANa₂ and 31 g KOH; the Fe solution uses water as a solvent, and comprises the following components on a basis of 1 L: 4.98 g FeSO₄·7H₂O and 1 mL H₂SO₄.

The technical solutions provided by the present invention will be described in detail below with reference to the examples, which, however, should not be construed as limiting the protection scope of the present invention.

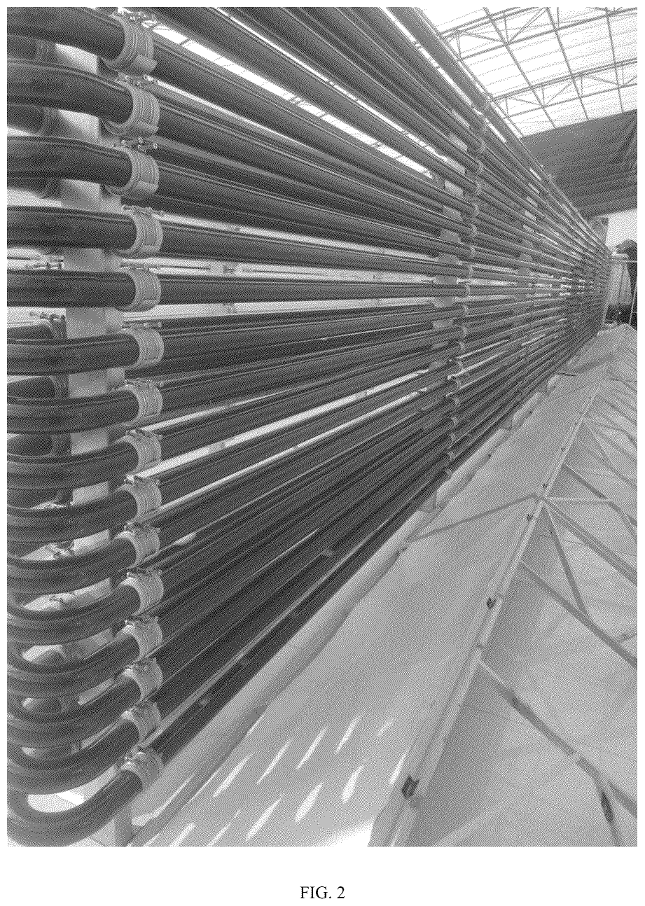

The structure of the light conversion film used in the following examples is shown in FIG. 2, and the height and the inclination angle can be adjusted; the film body of the light conversion film comprises an opaque film in the bottom layer, a light-reflecting film in the middle layer, and a light conversion film in the upper layer; the light conversion film in the upper layer comprises fluorescent powder, and the fluorescent powder can convert white light in sunlight into red light; the fluorescent powder is aluminum-based non-rare earth oxide type inorganic fluorescent powder described in Chinese Patent CN115261018A.

### Example 1

### Stage of Culture of Motile Vegetative Cells

The culture medium required by the culture of Haematococcus pluvialis motile vegetative cells.
1) The first culture medium was prepared using pure water with a conductivity of 10 S/m or less prepared by a water purifier as a solvent, comprising the following components in the following concentrations: 1.5 g/L NaNO₃, 0.75 g/L MgSO₄·7H₂O, 0.25 g/L NaCl, 0.75 g/L K₂HPO₄, 1.75 g/L KH₂PO₄, 0.25 g/L CaCl₂·2H₂O, and 0.114 g/L H₃BO₃ were added sequentially as required, and then 1 mL/L trace element liquid, 1.0 mL/L EDTA solution, and 1.0 mL/L Fe solution were added.

The preparation method for the trace element liquid was as follows: 8.82 g ZnSO₄·7H₂O, 1.44 g MnCl·4H₂O, 0.71 g MoO₃, 1.57 g CuSO₄·5H₂O, 0.49 g Co(NO₃)₂ 6H₂O, and 1.0 mL H₂SO₄ were added sequentially to 1 L water to prepare the trace element liquid.

The preparation method for the EDTA solution was as follows: 50 g EDTANa₂ and 31 g KOH were added sequentially to 1.0 L water to prepare the EDTA solution.

The preparation method for the Fe solution was as follows: 4.98 g FeSO₄·7H₂O and 1 mL H₂SO₄ were added sequentially to 1.0 L water to prepare the Fe solution.
2) the prepared first culture medium solution was poured into a glass container, sterilized at the high temperature of 120-130° C. and under the condition of 0.1-0.2 MPa for 10-20 min, taken out to be placed in a clean and sterile environment. When the temperature was reduced to 20-25° C., the Haematococcus pluvialis algae JNU35 was inoculated at an inoculation amount of 0.5 g/L on a dry weight basis.
3) the culture system inoculated with the Haematococcus pluvialis algae was added into a special cylindrical culture pipe (the diameter of an upper opening was 6 cm, the diameter of a lower opening was 12 cm, the bottom was in a conical shape, and a ventilation pipe was arranged in the culture pipe. Note: the ventilation pipe was 5 cm higher than the culture pipe, with the outer diameter of 6 mm and the inner diameter of 3 mm; the length of the sampling pipe was such that it reached below half of the liquid level of the culture medium, and the sampling pipe had an outer diameter of 3 mm and an inner diameter of 1 mm. The sampling pipe and the ventilation pipe were fixed by a rubber band. The distance between the ventilation pipe and the bottom of the culture pipe was ensured to be 2-3 mm, so that ventilation was ensured, and the sampling pipe was prevented from falling into the culture pipe. Finally, sterilization was performed with an aluminum foil wrapped on an autoclave), and the culture pipe was placed in a culture rack for indoor culture. The temperature of the culture room was observed during the culture period, and the temperature was controlled at 25° C. An air valve of carbon dioxide compressed air on the culture rack was observed, and 0.5-1% of carbon dioxide compressed air was controlled. The growth state of Haematococcus pluvialis on the culture rack was observed, and the light conversion film was used to enable the illumination to be 50-100 μmol/m². The illumination was controlled at 30 μmol/m² at the initial stage of the inoculation, and the illumination was adjusted depending on whether the algal liquid can be transparent at the concentration.
4) the inoculated algae liquid was cultured on a culture rack for 7 days as a cycle, the algae precipitated for 4 h using a precipitation method, the supernatant was discarded, a new first culture medium was added, and the operation after the inoculation was repeated until the dry weight of the Haematococcus pluvialis liquid reached 2.0 g/L or more;
   3.1 kg algae puree was finally collected in 600 L pipelines on Oct. 20, 2022, equivalent to 1.32 kg of dry weight, and the yield was 2.2 g/L.

Stage of culture of nonmotile vegetative cells

The motile vegetative cells obtained at the last stage were inoculated into a second culture medium for culturing for 14 days to obtain nonmotile vegetative cells with aged cell walls. The Haematococcus pluvialis was forced by factors such as high light, lack of nutrient elements and the like under the condition of introducing enough CO₂ to increase the thickness of the cell wall, and the characteristic that the cell wall is fragile was changed, so that the Haematococcus pluvialis was more easily adapted to the severe external environment.

The difference between the second culture medium and the first culture medium was only in that no sodium nitrate was added.

The second culture medium was prepared using pure water with a conductivity of 10 S/m or less prepared by a water purifier as a solvent, comprising the following components in the following concentrations: 1.5 g/L NaNO₃, 0.75 g/L MgSO₄·7H₂O, 0.25 g/L NaCl, 0.75 g/L K₂HPO₄, 1.75 g/L KH₂PO₄, 0.25 g/L CaCl₂·2H₂O, 0.114 g/L H₃BO₃, 1 mL/L trace element liquid, 1.0 mL/L EDTA solution, and 1.0 mL/L Fe solution.

The preparation method for the trace element liquid was as follows: 8.82 g ZnSO₄·7H₂O, 1.44 g MnCl·4H₂O, 0.71 g MoO₃, 1.57 g CuSO₄·5H₂O, 0.49 g Co(NO₃)₂ 6H₂O, and 1.0 mL H₂SO₄ were added sequentially to 1 L water to prepare the trace element liquid.

The preparation method for the EDTA solution was as follows: 50 g EDTANa₂ and 31 g KOH were added sequentially to 1.0 L water to prepare the EDTA solution.

The preparation method for the Fe solution was as follows: 4.98 g FeSO₄·7H₂O and 1 mL H₂SO₄ were added sequentially to 1.0 L water to prepare the Fe solution.

The illumination intensity was controlled at 100-125 μmol/m² using the light conversion film; the ambient temperature was 25° C.; 1-2% of carbon dioxide compressed air was controlled to be introduced.

### Stage of Induction of Generation of Astaxanthin

The nonmotile vegetative cells with aged cell walls were inoculated into a third culture medium at an inoculation amount of 0.5 g/L on a dry weight basis for outdoor culture for 10-12 days.

The third culture medium was prepared using pure water with a conductivity of 10 S/m or less prepared by a water purifier as a solvent, comprising the following components in the following concentrations: 0.5 g/L NaNO₃, 0.75 g/L MgSO₄·7H₂O, 1.5 g/L NaCl, 0.75 g/L K₂HPO₄, 1.75 g/L KH₂PO₄, 0.25 g/L CaCl₂·2H₂O, 0.114 g/L H₃BO₃, 1 mL/L trace element liquid, 1.0 mL/L EDTA solution, and 1.0 mL/L Fe solution.

The illumination intensity was controlled at 100-125 μmol/m² using the light conversion film; the ambient temperature was 25° C.; 1-2% of carbon dioxide compressed air was controlled to be introduced.

The astaxanthin content in Haematococcus pluvialis measured was 6% or more, and the culture was finished.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A culture method for high-concentration Haematococcus pluvialis, comprising the following steps:
1) inoculating a Haematococcus pluvialis algae into a first culture medium for culturing for 7-10 days, collecting algal cells, inoculating the algal cells into a new first culture medium for culturing until a dry weight concentration of the algal cells in a Haematococcus pluvialis liquid obtained by culturing is equal to or greater than 2.0 g/L, and collecting inoculated algal cells as motile vegetative cells,
wherein the first culture medium is a first improved Bold's Basal Medium (BBM) culture medium, and a concentration of NaNO₃ in the first improved BBM culture medium is 1.3-1.7 g/L, and an illumination intensity during the culturing of the motile vegetative cells is controlled at 30-100 μmol/m² by aid of a light conversion film, wherein the light conversion film comprises an aluminum-based non-rare earth oxide type inorganic fluorescent powder to convert white light into red light;
2) inoculating the motile vegetative cells obtained in step 1) into a second culture medium for culturing for 13-15 days to obtain nonmotile vegetative cells with aged cell walls,
wherein the second culture medium is a simplified BBM culture medium, and the simplified BBM culture medium does not comprise NaNO₃, and an illumination intensity during the culturing of the nonmotile vegetative cells is controlled at 100-125 μmol/m² by aid of a light conversion film, wherein the light conversion film comprises an aluminum-based non-rare earth oxide type inorganic fluorescent powder to convert white light into red light;
3) inoculating the nonmotile vegetative cells obtained in step 2) into a third culture medium for culturing for 10-14 days, inducing a generation of astaxanthin until a content of the astaxanthin in an algal powder is equal to or greater than 6%, and finishing the culturing,
wherein the third culture medium is a second improved BBM culture medium, and a concentration of NaCl in the second improved BBM culture medium is 1.3-1.7 g/L, and an illumination intensity during induction of the generation of the astaxanthin is controlled at 125-300 μmol/m² by aid of a light conversion film, wherein the light conversion film comprises an aluminum-based non-rare earth oxide type inorganic fluorescent powder to convert white light into red light.

2. The culture method according to claim 1, wherein the first culture medium uses pure water as a solvent and comprises the following components in the following concentrations: 1.3-1.7 g/L NaNO₃, 0.7-0.8 g/L MgSO₄·7H₂O, 0.2-0.3 g/L NaCl, 0.7-0.8 g/L K₂HPO₄, 1.7-1.8 g/L KH₂PO₄, 0.2-0.3 g/L CaCl₂·2H₂O, 0.11-0.12 g/L H₃BO₃, a 0.8-1.2 mL/L trace element liquid, a 0.8-1.2 mL/L EDTA solution, and a 0.8-1.2 mL/L Fe solution.

3. The culture method according to claim 1, wherein the second culture medium uses pure water as a solvent and comprises the following components in the following concentrations: 0.7-0.8 g/L MgSO₄·7H₂O, 0.2-0.3 g/L NaCl, 0.7-0.8 g/L K₂HPO₄, 1.7-1.8 g/L KH₂PO₄, 0.2-0.3 g/L CaCl₂·2H₂O, 0.11-0.12 g/L H₃BO₃, a 0.8-1.2 mL/L trace element liquid, a 0.8-1.2 mL/L EDTA solution, and a 0.8-1.2 mL/L Fe solution.

4. The culture method according to claim 1, wherein the third culture medium uses pure water as a solvent and comprises the following components in the following concentrations: 0.4-0.6 g/L NaNO₃, 0.7-0.8 g/L MgSO₄·7H₂O, 1.3-1.7 g/L NaCl, 0.7-0.8 g/L K₂HPO₄, 1.7-1.8 g/L KH₂PO₄, 0.2-0.3 g/L CaCl₂·2H₂O, 0.11-0.12 g/L H₃BO₃, a 0.8-1.2 mL/L trace element liquid, a 0.8-1.2 mL/L EDTA solution, and a 0.8-1.2 mL/L Fe solution.

5. (canceled)

6. The culture method according to claim 1, wherein during the culturing of the motile vegetative cells in step 1) an ambient temperature is 24-26° C.; a volume ratio of CO₂ introduced for the culturing to air introduced for the culturing is (0.5-1):(99.5-99).

7. The culture method according to claim 1, wherein during the culturing of the nonmotile vegetative cells in step 2) an ambient temperature is 24-26° C.; a volume ratio of CO₂ introduced for the culturing to air introduced for the culturing is (1-2):(99-98).

8. The culture method according to claim 1, wherein during a induction of the generation of the astaxanthin in step 3) an ambient temperature is 24-26° C.; a volume ratio of CO₂ introduced for the introduction to air introduced for the introduction is (1-2):(99-98).

9. (canceled)

10. The culture method according to claim 1, wherein the Haematococcus pluvialis algae is cultured using a pipeline culture; in a process of the pipeline culture of the motile vegetative cells, a bottom of a pipeline used is conical, and a ventilation pipe is arranged in the pipeline.

11. (canceled)

12. (canceled)

13. (canceled)

14. (canceled)
